(19) United States
(12) Patent Application Publication
(10) Pub. No.: US 2007/0020300 A1
(43) Pub. Date: Jan. 25, 2007

(54) RECREATIONAL WATER TREATMENT EMPLOYING SINGLET OXYGEN

(75) Inventors: Stephan M. Hubig, Maplewood, MN (US); Matthew J. Finley, Shakopee, MN (US); Robert D.P. Hei, Baldwin, WI (US)

Correspondence Address: IPLM GROUP, P.A., POST OFFICE BOX 18455, MINNEAPOLIS, MN, 55418, US

(73) Assignee: Ecolab Inc.

(21) Appl. No.: 11/534,146

(22) Filed: Sep. 21, 2006

Related U.S. Application Data
(62) Division of application No. 10/389,168, filed on Mar. 12, 2003.
Application No. 10/389,168 is a continuation-in-part of application No. 10/097,232, filed on Mar. 12, 2002.

(51) Int. Cl. A61K 9/00

(52) U.S. Cl. 424/400

(57) ABSTRACT

Recreational or process water can be treated by uniformly dissolving in the water a singlet oxygen-generating photosensitizer selected from the group consisting of condensed aromatic compounds, acridine dyes, coumarin dyes, crystal violet, fluorene derivatives, porphyrin derivatives, chlorins, thiazine dyes, thioketones, xanthene dyes, or by uniformly dissolving in the water a singlet oxygen-generating photosensitizer having an absorption maximum less than 659 nm and sufficiently strong coloration so that the presence of the photosensitizer can be visually detected in the water. Exposure of the photosensitizer and ambient or added oxygen to sufficient light or other activating energy causes the generation of singlet oxygen and the destruction or control of microorganisms.

### This application is a divisional of application Ser. No. 10/389,168 filed Mar. 12, 2003, which in turn is a continuation-in-part of application Ser. No. 10/097,232, filed Mar. 12, 2002.

### TECHNICAL FIELD

This invention relates to treatment of recreational waters such as fountains, ponds or pools, and to treatment of process waters such as factory process water and vehicle wash water.

### BACKGROUND

Singlet oxygen is an activated form of molecular oxygen. U.S. Pat. No. 4,740,308 describes a process for cleaning a fouled separation membrane by immersing the membrane in an aqueous inorganic peroxide solution followed by rinsing the membrane with an aqueous solution containing hypohalite ion. U.S. Pat. No. 5,679,661 describes low hue phthalocyanine and naphphthalocyanine photosensitizing compounds said to be useful, inter alia, for treating swimming baths and for making water potable or for maintaining water in a potable state. The photosensitizing compound is said to generate singlet oxygen in the presence of light and oxygen. Moore, P., “Lethal Weapon”, New Scientist, 158, No. 2130, pp. 40-43 (Apr. 18, 1998) mentions that singlet oxygen might be formed in the presence of photosensitizers bound to polymer beads or trapped inside a porous material and used to purify water. U.S. Pat. Nos. 4,525,255 and 4,609,444 mention that singlet oxygen might be added to various compounds using a photosensitizer bound to a floating polymer bead and thereby carrying out certain aqueous chemical reactions.

### SUMMARY OF INVENTION The present invention provides, in one aspect, a method for treating recreational waters or process waters comprising:

a. providing a body of recreational or process water;
b. uniformly dissolving therein an antimicrobially effective amount of at least one singlet oxygen-generating photosensitizer selected from the group consisting of condensed aromatic compounds, acridine dyes, coumarin dyes, crystal violet, fluorene derivatives, porphyrin derivatives, chlorins, thiazine dyes, thioketones, xanthene dyes; and
c. exposing the photosensitizer and ambient or added oxygen to sufficient light or other activating energy to cause generation of singlet oxygen and destruction or reduction in the population of at least one microorganism of interest in such water.

The present invention provides, in yet another aspect, a method for treating recreational waters or process waters comprising:
a. providing a body of recreational or process water;
b. dissolving therein an antimicrobially effective amount of at least one singlet oxygen-generating photosensitizer having an absorption maximum less than 659 nm and sufficiently strong coloration so that the presence of the photosensitizer can be visually detected in the water; and
c. exposing the photosensitizer and ambient or added oxygen to sufficient light or other activating energy to cause generation of singlet oxygen and destruction or reduction in the population of at least one microorganism of interest in such water.

The present invention provides, in another aspect, a body of treated recreational or process water having uniformly dissolved therein an antimicrobially effective amount of at least one singlet oxygen-generating photosensitizer selected from the group consisting of condensed aromatic compounds, acridine dyes, coumarin dyes, crystal violet, fluorene derivatives, porphyrin derivatives, chlorins, thiazine dyes, thioketones, xanthene dyes.

The present invention provides, in another aspect, a body of treated recreational or process water having dissolved therein an antimicrobially effective amount of at least one singlet oxygen-generating photosensitizer having an absorption maximum less than 659 nm and sufficiently strong coloration so that the presence of the photosensitizer can be visually detected in the water.

The present invention provides, in yet another aspect, a kit for treating a body of recreational or process water comprising a composition containing a singlet oxygen-generating photosensitizer selected from the group consisting of condensed aromatic compounds, acridine dyes, coumarin dyes, crystal violet, fluorene derivatives, porphyrin derivatives, chlorins, thiazine dyes, thioketones, xanthene dyes and instructions for (a) uniformly dissolving the composition in the body of water and (b) exposing the photosensitizer to sufficient light or other activating energy to cause generation of singlet oxygen and a reduction in the population in such water of a microbe of interest.

The present invention provides, in yet another aspect, a kit for treating recreational or process water comprising at least one singlet oxygen-generating photosensitizer having an absorption maximum less than 659 nm and sufficiently strong coloration so that the presence of an antimicrobially effective amount of the photosensitizer can be visually detected in the water, together with instructions for dissolving the photosensitizer in recreational or process water and exposing the water to sufficient light or other activating energy to cause generation of singlet oxygen and a reduction in the population in such water of a microbe of interest.

The compositions and methods of the invention can significantly reduce or control microorganism populations in recreational and process waters. A variety of microorganisms can be reduced or controlled. For embodiments of the invention employing strongly colored photosensitizers, the coloration of the photosensitizer and its presence as a dissolved species make it easy to ascertain through visual inspection that the photosensitizer is present.

### DETAILED DESCRIPTION

As used in this invention, the term “recreational water” refers to water for use in fountains, ponds (including fish or duck ponds and the like) or pools (including swimming pools, spas and the like), but not intended for discharge as effluent. The term “process water” refers to water for use in factory processes, vehicle wash facilities and the like, but not intended for discharge as effluent. The term “treatable water” refers to recreational or process water to be treated in order to reduce or control microorganism levels.

The term “antimicrobially effective amounts” when used in respect to dissolved oxygen and a dissolved photosensitizer refers to amounts sufficient to destroy or suppress the growth of at least one microorganism of interest in treatable water. In most instances, using the procedure set out in Germicidal and Detergent Sanitizing Action of Disinfectants, Official Methods of Analysis of the Association of Official Analytical Chemists, paragraph 960.09 and applicable sections, 15th Edition, 1990 (EPA Guideline 91-2), the methods and kits of the invention provide greater than a 90% reduction (greater than a 1-log order reduction), preferably greater than a 99% reduction (greater than a 2-log order reduction), more preferably greater than a 99.99% reduction (greater than a 4-log order reduction), and most preferably greater than a 99.999% reduction (greater than a 5-log order reduction) in one or more targeted species of interest (e.g., the bacteria S. aureus, P. aeruginosa, E. coli or S. typhi, or the fungus Aspergillus sp.) within 30 minutes at ambient temperature. Preferably, the antimicrobial compositions of the invention also provide greater than a 99% reduction (greater than a 2-log order reduction), more preferably greater than a 99.99% reduction (greater than a 4-log order reduction), and most preferably greater than a 99.999% reduction (greater than a 5-log order reduction) in the population of two or more targeted species of interest under such conditions. Because in their broadest sense these definitions for antimicrobial activity are different from some current governmental regulations, the use in connection with this invention of the term “antimicrobially effective” is not intended to indicate compliance with any particular governmental standard for antimicrobial activity.

The term “ambient or added oxygen” refers to molecular oxygen already present in a body of treatable water (e.g., as dissolved oxygen) or added thereto (e.g., by bubbling air of oxygen into the treatable water). The term “singlet oxygen-generating” when used in respect to a photosensitizer or composition refers to a photosensitizer or composition that when subjected to a suitable internal or external activating agent or activating energy in the presence of added or ambient oxygen will provide an antimicrobially effective amount of singlet oxygen in treatable water. The term “in situ” refers to the generation of singlet oxygen in a body of treatable water. The term “strongly colored” when used in connection with a photosensitizer refers to the color of a solution of pure water containing the dissolved photosensitizer. The term “can be visually detected” when used in respect to water containing a dissolved photosensitizer refers to a coloration that is sufficiently intense so that the color can be seen by placing a 1 ppm aqueous solution of the photosensitizer in a 100 ml beaker in front of a white background and comparing it to an otherwise similar sample of water that does not contain the photosensitizer.

The photosensitizer can generate singlet oxygen when exposed to light in the presence of ambient or added oxygen according to the following reactions:
Photosensitizer+hv→Photosensitizer* (1)
Photosensitizer*+O₂→O₂*(singlet oxygen) (2)
In this mechanism, energy transfer from an excited state (typically a triplet state) of the photosensitizer to molecular oxygen generates singlet oxygen molecules. The photosensitizer preferably has a sufficiently long lifetime in its excited state so that sufficient molecular oxygen will be encountered and sufficient singlet oxygen generated by the excited photosensitizer. Preferably this lifetime is at least about 2 microseconds, more preferably at least about 10 microseconds, and most preferably at least about 100 microseconds.

Strongly colored photosensitizers are especially preferred for use in the invention, e.g., red, orange, yellow, green, blue, indigo or violet photosensitizers that can be visually detected as described above. Blue and indigo photosensitizers are particularly preferred for use in recreational waters. In some instances the photosensitizer color may be chosen to or may happen to match or mask the color of algae or other opacity-inducing microorganisms that may be present in treatable water. In such cases the presence of an antimicrobially effective amount of the photosensitizer may be determined by noting the visually detectable color of the photosensitizer and by noting that the treated water remains transparent. Photosensitizers having an absorption maximum below 659 nm are also preferred. Low hue photosensitizers such as those described in U.S. Pat. No. 5,679,661 are designed to have maximum light absorption at a relatively high wavelength (e.g., 660 nm or more), and thus it can be difficult to determine through mere inspection of treated water whether a sufficient amount of such a photosensitizer is present. Photosensitizers bound to a polymer bead or trapped inside a porous material such as those mentioned or described in the New Scientist article or in U.S. Pat. Nos. 4,525,255 and 4,609,444 are designed to be separated from treated water, and thus it can likewise be difficult to determine through mere inspection of the treated water whether a sufficient amount of such a photosensitizer has been employed.

Preferred photosensitizers for use in the invention include condensed aromatic compounds (e.g., anthracene, naphthalene, pyrene and rubrene), acridine dyes (e.g., acridine orange), coumarin dyes (e.g., thiocoumarin), crystal violet, fluorene derivatives (e.g., fluorene and fluorenones), porphyrin derivatives (e.g., copper porphyrin, zinc tetraphenylporphyrin tetrasulfonate, and chlorins such as 5,10,15,20-Tetrakis(m-hydroxyphenyl)chlorin, thiazine dyes (e.g., methylene blue and toluidine blue), thioketones, xanthene dyes (e.g., eosin B, eosin Y, fluorescein and rose bengal) and the like, as well as mixtures thereof. Preferred strongly colored photosensitizers having an absorption maximum less than 659 nm and sufficiently strong coloration so that the presence of the photosensitizer can be visually detected in the water include condensed aromatic compounds (e.g., rubrene, λmax˜475-520 nm), acridine dyes (e.g., acridine orange, λmax 489 nm), coumarin dyes (e.g., thiocoumarin), crystal violet (λmax 588 nm), fluorene derivatives (e.g., fluorenones), phthalocyanines and naphthalocyanines having an absorption maximum below 659 nm, porphyrin derivatives (e.g., copper porphyrin, zinc tetraphenylporphyrin tetrasulfonate, and chlorins such as 5,10,15,20-Tetrakis(m-hydroxyphenyl)chlorin, thiazine dyes (e.g., toluidine blue, λmax 626 nm), thioketones, xanthene dyes (e.g., eosin B, λmax 514 nm; eosin Y, λmax 517 nm; fluorescein, λmax 496 nm and rose bengal, λmax 548 nm) and the like, as well as mixtures thereof. Those skilled in the art will recognize that photosensitizers other than those listed above may be employed, so long as the photosensitizer functions adequately as a singlet oxygen-generating agent under the desired conditions of use.

Preferably the photosensitizer is not toxic or irritating to humans, and most preferably is regarded as safe for use in food or for ingestion by humans. Photosensitizers such as acridine orange, eosin, fluorene, fluorenone, fluorescein, methylene blue, toluidine blue rose bengal, chlorins such as 5,10,15,20-Tetrakis(m-hydroxyphenyl)chlorin, thiocoumarin, toluidine blue and zinc tetraphenylporphyrin tetrasulfonate are preferred, with methylene blue, toluidine blue and rose bengal being especially preferred. The treatable water preferably contains at least about 0.1 ppm photosensitizer, more preferably about 0.1 to about 50 ppm.

The above-mentioned reaction (1) requires light. A variety of light sources can be employed. The light source is preferably sunlight or room light such as incandescent or fluorescent light. Coherent or narrow spectrum sources such as lasers and light emitting diodes, flash tubes, arc lamps, mercury vapor lamps, tungsten halide lamps, sodium lamps and other sources that will be familiar to those skilled in the art can also be employed, so long as the chosen light source provides the desired degree of singlet oxygen generation and safety under the desired conditions of use. When strongly colored photosensitizers are employed, reaction (1) typically will take place in visible light. Faster reaction may occur when the light source provides both visible light and UV energy. For many applications, the light from a typical 60 W to 100 W incandescent bulb, placed within a few centimeters of the desired target area (e.g., the surface of a body of treatable water), will provide sufficient illumination. The intensity of illumination can vary within wide limits, depending in part on the type and concentration of photosensitizer. The exposure time can also vary, with exposure times of a few minutes up to a few hours being preferred and exposure times of a few minutes to about one hour being more preferred. As those skilled in the art will appreciate, use of lower intensity illumination or a greater distance between the light source and the treatable water will typically require a longer exposure time.

The above-mentioned reaction (2) requires the presence or addition of ambient or added oxygen. Ambient or added oxygen can be provided in a variety of ways, e.g., from air near the treatable water, from air or oxygen that is bubbled into the treatable water, or from air or oxygen already present in the treatable water. Relatively small amounts of ambient or added oxygen can be employed. For example, the amount of dissolved molecular oxygen that typically is present in aerated water (about 10⁻⁴ M) will usually suffice.

The photosensitizer can be added neat to treatable water or in combination with other ingredients in a treating composition. Suitable treating compositions can be a solid or liquid. If a solid, the treating composition can have a powdered form, or if desired can be shaped or molded into a suitable pellet, block or other convenient shape, e.g., for use in a dispenser. If in liquid form, the treating composition can be aqueous (that is, primarily containing water) or non-aqueous (that is, primarily containing a liquid or liquids other than water). Once added, the photosensitizer should uniformly dissolve in the treatable water, rather than being encapsulated in beads or other carriers that discourage uniform dissolution of the photosensitizer in the treatable water.

The treating composition optionally can contain at least one chelating agent. The presence of a chelating agent can synergistically increase the level of antimicrobial activity. A variety of chelating agents can be employed, including tris(hydroxymethyl)aminomethane (“TRIS”), amino carboxylates (e.g., amino succinates or acetates such as ethylenediamine disuccinate, ethylenediamine tetraacetate (“EDTA”), N-hydroxyethylethylenediamine triacetate; ethylenediamine tetraproprionates; triethylenetetraamine hexacetate; or diethylenetriamine pentaacetate); nitrilotriacetates; ethanoldiglycines; amino phosphonates; polysubstituted aromatic chelating agents; other chelating agents that will be familiar to those skilled in the art; as well as mixtures thereof or salts thereof (e.g., alkali metal, ammonium, or substituted ammonium salts). Preferably, the treating composition contains about 0.1 to about 1000 ppm, more preferably about 1 to about 100 ppm chelating agent based on the total weight of the treating composition. An approximately 10:1 molar mixture of EDTA and TRIS at a use concentration of about 100 to about 5000 ppm is especially preferred.

The treating composition optionally can contain at least one surfactant. The presence of a surfactant can also synergistically increase the level of antimicrobial activity. A variety of surfactants can be employed, including anionic, cationic, nonionic, amphoteric or zwitterionic surfactants such as alkyl benzene sulfonates, alkyl sulfates, unsaturated sulfates, alkyl alkoxy sulfates, alkyl alkoxy carboxylates, glycerol ethers, alkyl polyglycosides and their corresponding sulfated polyglycosides, alpha-sulfonated fatty acid esters, alkyl ethoxylates, alkyl phenol alkoxylates, betaines, sulfobetaines, N-alkyl polyhydroxy fatty acid amides, N-alkoxy polyhydroxy fatty acid amides, other surfactants that will be familiar to those skilled in the art, and mixtures thereof. Preferably the treating composition contains about 1 ppm to about 10 wt. %, more preferably about 10 ppm to about 1 wt. % surfactant based on the total weight of the treating composition. A use concentration in treatable water of about 0.1 wt. to about 1 wt. % is especially preferred.

Those skilled in the art will appreciate that the treating composition can include a variety of optional adjuvants. For example, the treating composition can contain additional antimicrobial agents (e.g., carboxylic acids, diacids, or triacids such as butyric acid, heptanoic acid, octanoic acid, nonanoic acid, decanoic acid, salycic acid, mandelic acid, succinic acid, adipic acid, glutaric acid, EDTA and citric acid), carboxylic esters (e.g., p-hydroxy alkyl benzoates and alkyl cinnamates), sulfonic acids (e.g., dodecylbenzene sulfonic acid), iodo-compounds or active halogen compounds (e.g., iodine, interhalides, polyhalides, metal hypochlorites, hypochlorous acid, metal hypbromites, hypobromous acid, chloro- and bromo-hydantoins, chlorine dioxide and sodium chlorite), isolated or equilibrium derived or isolated peracids such as chloroperbenzoic acids, peracetic acid, perheptanoic acid, peroctanoic acid, perdecanoic acid, performic acid, percitric acid, perglycolic acid, perlactic acid, perbenzoic acid, and monoester peracids derived from diacids or diesters (e.g., such as adipic, succinic, glutaric or malonic acid and mixtures thereof), organic peroxides including benzoyl peroxide, alkyl benzoyl peroxides, and mixtures thereof, phenolic derivatives (e.g., o-phenyl phenol, o-benzyl-p-chlorophenol, tert-amyl phenol and C₁-C₆ alkyl hydroxy benzoates), quaternary ammonium compounds (e.g., alkyldimethylbenzyl ammonium chloride or dialkyldimethyl ammonium chloride), other antimicrobial agents that will be familiar to those skilled in the art, and mixtures thereof. Other adjuvants include additional colorants, fillers, buffers, electrolytes, foam inhibitors or foam generators, plasticizers, viscosity modifiers and solvents. The amounts and types of such adjuvants will be apparent to those skilled in the art.

Dissolution of the photosensitizer or treating composition in a body of treatable water can be carried out using any suitable technique including pouring a liquid or powdered form of the photosensitizer or treating composition onto or mixing it into the treatable water, adding a sachet containing photosensitizer or treating composition to the treatable water, spray application, drip application, and other methods that will be apparent to those skilled in the art. Dissolution can be aided with stirring, agitation or aeration. Additional photosensitizer or treating composition can be added on a regular basis or whenever needed to maintain a desired photosensitizer level, with the visibly detected loss of color in the treatable water providing a good indication that additional photosensitizer or treating composition should be added or that additional oxygen is required.

The invention is further illustrated in the following non-limiting examples, in which all parts and percentages are by weight unless otherwise indicated.

### EXAMPLE 1

The Antimicrobial Effect of Various Singlet Oxygen-Generating Photosensitizers

Several photosensitizers were dissolved in aerated sterile water to form test solutions, injected with cultures of S. aureus or P. aeruginosa and evaluated to determine the extent to which the microbial population was reduced under dark conditions and when exposed to a 90 W lamp located about 30 cm above the test solution. For test solutions exposed to the 90 W lamp, the solution temperature rose somewhat above room temperature but remained below 41° C. The results are set out below in Table 1, with the photosensitizer being referred to in the table as a “dye”.

TABLE 1
The Antimicrobial Effect of Various Photosensitizers

| | | | | Microbial Log | Microbial Log |
|---|---|---|---|---|---|
| | | | | Reduction (30 | Reduction |
| Run No. | Dye | Dye Conc. | Organism | Minute) | (60 Minute) |
| 1-1 | Toluidine | 3.1 ppm | S. aureus | 0.1 | 0.1 |
| (Control | Blue | | P. aeruginosa | <0.6 | <0.6 |
| w/ no light) | | | | | |
| 1-2 | Toluidine | 3.1 ppm | S. aureus | 2.8 | 5.4 |
| (90 W | Blue | | P. aeruginosa | 2.7 | 5.11 |
| Lamp) | | | | | |
| 1-3 | Rose | 10 ppm | S. aureus | 3.0 | >6.4 |
| (90 W | Bengal | | P. aeruginosa | 0.4 | 0.7 |
| Lamp) | | | | | |
| 1-4 | Acridine | 6.3 ppm | S. aureus | 4.7 | >6.4 |
| (90 W | Orange | | P. aeruginosa | 5.2 | 5.0 |
| Lamp) | | | | | |
| 1-5 | 50/50 | 16.5 ppm | S. aureus | 6.1 | 6.4 |
| (90 W | mixture of | | P. aeruginosa | 0.3 | 0.5 |
| Lamp) | Rose | | | | |
| | Bengal/ | | | | |
| | Acridine | | | | |
| | Orange | | | | |
| 1-6 | Methylene | 4.5 ppm | S. aureus | 4.3 | >6.4 |
| (90 W | Blue | | P. aeruginosa | 0.71 | 1.64 |
| Lamp) | | | | | |

Each of the test solutions was strongly colored. The presence of the photosensitizer could readily be visually detected by holding a beaker containing the test solution in front of a white background alongside a beaker containing pure water. Comparison of the control run (Run No. 1-1) to the test solution containing a dissolved photosensitizer exposed to light (Run Nos. 1-2 through 1-7) shows that microbial reduction improved significantly (amounting in some cases to several orders of magnitude) when the photosensitizer, oxygen and light were all present.

### EXAMPLE 2

pH Effects

A pH kill rate profile for toluidine blue photosensitizer was determined in aqueous solution by dissolving 1.0 ppm of toluidine blue in aerated sterile water adjusted to various pH levels, injecting cultures of E. coli or S. aureus and exposing the resulting test solutions for 15, 30 and 60 minutes to light from a 90 W lamp located about 30 cm above the test solution. Antimicrobial activity was evaluated using the method of Example 1. The results are set out below in Table 2.

TABLE 2
Antimicrobial pH Profile

| | | | Microbial | Microbial | Microbial |
|---|---|---|---|---|---|
| | | | Log | Log | Log |
| | | | Reduction | Reduction | Reduction |
| Run | Solution | | (15 | (30 | (60 |
| No. | pH | Organism | Minutes) | Minutes) | Minutes) |
| 2-1 | 4 | E. coli | 0.4 | 0.8 | 6.2 |
| | | S. aureus | 0.8 | 2.8 | >8.2 |
| 2-2 | 6 | E. coli | 6.7 | 8.0 | >8.0 |
| | | S. aureus | 6.6 | >8.2 | >8.2 |
| 2-3 | 8 | E. coli | 7.7 | >8.0 | >8.0 |
| | | S. aureus | >8.2 | >8.2 | >8.2 |
| 2-4 | 10 | E. coli | >8.0 | >8.0 | >8.0 |
| | | S. aureus | 4.9 | >8.2 | >8.2 |

The results in Table 2 show that toluidine blue photosensitizer worked most rapidly at neutral to alkaline pH levels. However, longer exposure times provided good microbial reduction at all pH levels.

### EXAMPLE 3

The Effect of Oxygen on Microbial Reduction

The effect of adding oxygen to treatable water containing a photosensitizer was evaluated by dissolving 1.3 ppm of toluidine blue photosensitizer in sterile water, injecting cultures of E. coli or S. aureus, covering the resulting test solutions with a barrier film, and bubbling nitrogen or oxygen gas through the test solutions while exposing the test solutions for 15, 30 and 60 minutes to light from a 90 W lamp located about 30 cm above the test solution. Exposure to nitrogen gas tended to carry away oxygen above the test solution and to reduce the concentration of dissolved oxygen to negligible levels. Exposure to oxygen gas tended to increase the amount of dissolved oxygen. The results are set out below in Table 3.

TABLE 3
Exposure to Nitrogen or Oxygen Gas

| | | Bubbling/ | Microbial | Microbial |
|---|---|---|---|---|
| | | Exposure | Log | Log |
| | | Time | Reduction, | Reduction, |
| Run No. | Condition | (Minutes) | E. coli | S. aureus |
| 3-1 | Bubbled Nitrogen | 15 | 0.4 | 0.2 |
| | | 30 | 0.5 | 0.3 |
| | | 60 | 1.7 | 2.5 |
| 3-2 | Bubbled Oxygen | 15 | >7.2 | 3.4 |
| | | 30 | >7.2 | 4.8 |
| | | 60 | >7.2 | >6.8 |

The results in Table 3 show the beneficial effect of adding oxygen to the test solutions and the negative effect of its exclusion. The blue photosensitizer coloration faded very rapidly when nitrogen was bubbled into the test solution (caused by a photoreduction reaction that occurs in the absence of oxygen), but the color intensity returned after re-oxidation.

### EXAMPLE 4

Antifungal Efficacy

Varying amounts of toluidine blue photosensitizer were dissolved in aerated sterile water or a 0.5% aerated solution of “TRIS-EDTA” (a 1:10 molar mixture of tris(hydroxymethyl)aminomethane and ethylenediamine tetraacetate), injected with a culture of the fungus Aspergillus sp. and exposed for times ranging from 60 to 240 minutes to light from a 90 W lamp located about 30 cm above the test solution. The results are set out below in Table 4.

TABLE 4
Antifungal Effect

| | | | Exposure | Microbial Log |
|---|---|---|---|---|
| Run | Toluidine | | Time | Reduction, |
| No. | Blue Conc. | Test Solution | (Minutes) | Aspergillus sp. |
| 4-1 | 1.0 ppm | Sterile Water | 60 | 0.1 |
| 4-2 | 4.0 ppm | Sterile Water | 60 | 0.3 |
| 4-3 | 5.6 ppm | Sterile Water | 60 | 0.1 |
| | | | 120 | 0.6 |
| | | | 240 | 3.2 |
| 4-4 | 5.6 ppm | 0.5 wt % TRIS- | 60 | 2.6 |
| | | EDTA | | |

The results in Table 4 show that a positive antifungal effect can be achieved under a variety of conditions. Addition of TRIS-EDTA gave a synergistic increase in antifungal efficacy.

### EXAMPLE 5

Chelant Addition

Using the method of Example 4, varying amounts of toluidine blue photosensitizer were dissolved in a 0.5% solution of TRIS-EDTA in water, injected with cultures of S. aureus or P. aeruginosa and exposed for 30 minutes to light from a 90 W lamp located about 30 cm from the test surface. The results are set out below in Table 5.

TABLE 5
Antimicrobial Improvement Using a Chelant

| | | | | Microbial |
|---|---|---|---|---|
| | | | | Log |
| Run | Toluidine | | | Reduction |
| No. | Blue Conc. | Test Solution | Organism | (30 Minute) |
| 10-1 | 0.0 ppm | 0.5 wt % Tris- | S. aureus | 0.0 |
| | | EDTA | | |
| 10-3 | 5.6 ppm | 0.5 wt % Tris- | S. aureus | >6.8 |
| | | EDTA | | |
| 10-6 | 5.6 ppm | 0.5 wt % Tris- | P. aeruginosa | >7.4 |
| | | EDTA | | |

The results in Table 5 and Table 4 show that whereas the TRIS-EDTA solution has no biocidal properties by itself, it decisively does not hinder and can synergistically assist in broad-spectrum antimicrobial water treatment.

### EXAMPLE 6

Effect of UV (Sunlight)

Varying amounts of toluidine blue or rose bengal photoinitiator were dissolved in aerated sterile water, injected with cultures of S. aureus or P. aeruginosa and exposed for 30 or 60 minutes to sunlight with and without a glass cover over the test solution. The glass cover screened out radiation below 300 nm. The results are set out below in Table 6.

TABLE 6
The Antimicrobial Effect of UV (Sunlight)

| | | | | Microbial | Microbial |
|---|---|---|---|---|---|
| | | | | Log | Log |
| | | | | Reduction | Reduction |
| Run | | Dye | | (30 | (60 |
| No. | Dye | Conc. | Organism | Minute) | Minute) |
| 11-1 | Toluidine | 1.1 ppm | S. aureus | 4.3 | >6.3 |
| | Blue | | P. aeruginosa | >7.2 | >7.2 |
| 11-2 | Toluidine | 1.1 ppm | S. aureus | 3.9 | >6.3 |
| | Blue under | | P. aeruginosa | >7.2 | >7.2 |
| | glass | | | | |
| 11-3 | Rose | 3.3 ppm | S. aureus | >6.3 | >6.3 |
| | Bengal | | P. aeruginosa | >7.2 | >7.2 |
| 11-4 | Rose | 3.3 ppm | S. aureus | >6.3 | >6.3 |
| | Bengal | | P. aeruginosa | >7.2 | >7.2 |
| | under | | | | |
| | glass | | | | |

The results in Table 6 show that test samples covered with glass exhibited antimicrobial efficacy that under these conditions was similar to that exhibited by samples that were not covered with glass. In general however, enhanced antimicrobial efficacy will generally be obtained when the photosensitizer is strongly colored in the visible range and treatable water containing the dissolved photoinitiator and oxygen is exposed to a broad spectrum of light containing both visible and UV radiation.

Various modifications and alterations of this invention will be apparent to those skilled in the art without departing from the scope and spirit of the invention, and are intended to be within the scope of the following claims.

1. A process for treating recreational waters or process waters comprising:
a. providing a body of recreational or process water;
b. uniformly dissolving therein an antimicrobially effective amount of at least one singlet oxygen-generating photosensitizer selected from the group consisting of condensed aromatic compounds, acridine dyes, coumarin dyes, crystal violet, fluorene derivatives, porphyrin derivatives, chlorins, thiazine dyes, thioketones and xanthene dyes, and without encapsulating the photosensitizer in beads or other carriers that discourage uniform dissolution of the photosensitizer in water; and
c. exposing the photosensitizer and ambient or added oxygen to sufficient light or other activating energy to cause generation of singlet oxygen and destruction or reduction in the population of at least one microorganism of interest in such water.

2. A process according to claim 1 wherein the photosensitizer is at least one of acridine orange, eosin, fluorene, fluorenone, fluorescein, thiocoumarin, zinc tetraphenylporphyrin tetrasulfonate or a mixture thereof.

3. A process according to claim 1 wherein the photosensitizer is at least one of methylene blue, toluidine blue, rose bengal or a mixture thereof.

4. A process according to claim 1 wherein about 0.1 to about 50 ppm of the photosensitizer is dissolved in the body of water.

5. A process according to claim 1 further comprising dissolving a chelating agent or a surfactant in the body of water.

6. A process according to claim 1 wherein greater than a 4-log order reduction in one or more of the bacteria S. aureus, P. aeruginosa, E. coli or S. typhi, or the fungus Aspergillus sp. is obtained within 30 minutes after exposing the dissolved photosensitizer to light.

7. A process according to claim 1 wherein greater than a 5-log order reduction in one or more of the bacteria S. aureus, P. aeruginosa, E. coli or S. typhi, or the fungus Aspergillus sp. is obtained within 30 minutes after exposing the dissolved photosensitizer to light.

8. A process according to claim 1 wherein the body of water comprises a fountain, pond or pool.

9. A process according to claim 1 wherein the body of water comprises factory process water or vehicle wash water.

10. A process for treating recreational waters or process waters comprising:
a. providing a body of recreational or process water;
b. dissolving therein an antimicrobially effective amount of at least one singlet oxygen-generating photosensitizer having an absorption maximum less than 659 nm and sufficiently strong coloration so that the presence of the photosensitizer can be visually detected in the water, and without encapsulating the photosensitizer in beads or other carriers that discourage uniform dissolution of the photosensitizer in water; and
c. exposing the photosensitizer and ambient or added oxygen to sufficient light or other activating energy to cause generation of singlet oxygen and destruction or reduction in the population of at least one microorganism of interest in such water.

11. A process according to claim 10 wherein the photosensitizer is at least one of a condensed aromatic compound, coumarin dye, crystal violet, fluorenone, phthalocyanine, naphthalocyanine, porphyrin derivative, chlorin, thiazine dye, thioketone, xanthene dye or mixture thereof.

12. A process according to claim 10 wherein the photosensitizer is at least one of acridine orange, eosin, fluorenone, fluorescein, thiocoumarin, zinc tetraphenylporphyrin tetrasulfonate or a mixture thereof.

13. A process according to claim 10 wherein the photosensitizer is at least one of toluidine blue, rose bengal or a mixture thereof.

14. A process according to claim 10 wherein about 0.1 to about 50 ppm of the photosensitizer is dissolved in the body of water.

15. A process according to claim 10 further comprising dissolving a chelating agent or a surfactant in the body of water.

16. A process according to claim 10 wherein greater than a 4-log order reduction in one or more of the bacteria S. aureus, P. aeruginosa, E. coli or S. typhi, or the fungus Aspergillus sp. is obtained within 30 minutes after exposing the dissolved photosensitizer to light.

17. A process according to claim 10 wherein greater than a 5-log order reduction in one or more of the bacteria S. aureus, P. aeruginosa, E. coli or S. typhi, or the fungus Aspergillus sp. is obtained within 30 minutes after exposing the dissolved photosensitizer to light.

18. A process according to claim 10 wherein the body of water comprises a fountain, pond or pool.

19. A process according to claim 10 wherein the body of water comprises factory process water or vehicle wash water.

20. A body of treated recreational or process water having uniformly dissolved therein an antimicrobially effective amount of at least one singlet oxygen-generating photosensitizer selected from the group consisting of condensed aromatic compounds, acridine dyes, coumarin dyes, crystal violet, fluorene derivatives, porphyrin derivatives, chlorins, thiazine dyes, thioketones and xanthene dyes.

21. A body of water according to claim 20 wherein the photosensitizer is at least one of acridine orange, eosin, fluorene, fluorenone, fluorescein, thiocoumarin, zinc tetraphenylporphyrin tetrasulfonate or a mixture thereof.

22. A body of water according to claim 20 wherein the photosensitizer is at least one of methylene blue, toluidine blue, rose bengal or a mixture thereof.

23. A body of water according to claim 20 comprising about 0.1 to about 50 ppm of the photosensitizer.

24. A body of water according to claim 20 further comprising a chelating agent or surfactant.

25. A body of treated recreational or process water having dissolved therein an antimicrobially effective amount of at least one singlet oxygen-generating photosensitizer having an absorption maximum less than 659 nm and sufficiently strong coloration so that the presence of the photosensitizer can be visually detected in the water.

26. A body of water according to claim 25 wherein the photosensitizer is at least one of a condensed aromatic compound, coumarin dye, crystal violet, fluorenone, phthalocyanine, naphthalocyanine, porphyrin derivative, chlorin, thiazine dye, thioketone, xanthene dye or mixture thereof.

27. A body of water according to claim 25 wherein the photosensitizer is at least one of acridine orange, eosin, fluorenone, fluorescein, thiocoumarin, zinc tetraphenylporphyrin tetrasulfonate or a mixture thereof.

28. A body of water according to claim 25 wherein the photosensitizer is at least one of toluidine blue, rose bengal or a mixture thereof.

29. A body of water according to claim 25 comprising about 0.1 to about 50 ppm of the photosensitizer.

30. A body of water according to claim 25 further comprising a chelating agent or surfactant.
